(19) United States
(12) Patent Application Publication
(10) Pub. No.: US 2021/0059643 A1
(43) Pub. Date: Mar. 4, 2021

(54) METHOD AND SYSTEM FOR SHEAR WAVE ELASTOGRAPHY AND MEDIUM STORING CORRESPONDING PROGRAM

(71) Applicant: GE Precision Healthcare LLC, Wauwatosa, WI (US)

(72) Inventors: Gang LIU, Wuxi (CN); Wei JIANG, Wuxi (CN); Chengyang DU, Wuxi (CN); Xiaodong HAN, Wuxi (CN); Feng WU, Wuxi (CN)

(21) Appl. No.: 16/985,442

(22) Filed: Aug. 5, 2020

(30) Foreign Application Priority Data
Sep. 4, 2019 (CN) 201910833221.X

(51) Int. Cl. A61B 8/08; A61B 8/00; G01S 7/52

(52) U.S. Cl. CPC A61B 8/485; A61B 8/463; G01S 7/52085; A61B 8/5223; G01S 7/52042; A61B 8/085

(57) ABSTRACT

The present invention relates to a method and system for shear wave elastography and a medium storing a corresponding program. The method comprises: obtaining an initial image of an object; defining a region of interest in the initial image; performing shear wave elastography on the object at a plurality of different vibration frequencies, and generating a plurality of images corresponding to the plurality of different vibration frequencies; and determining an image corresponding to a specific vibration frequency in the plurality of different vibration frequencies as an optimized image based on the region of interest. The present invention further provides a corresponding system and a medium storing a corresponding program.

### CROSS-REFERENCE TO RELATED APPLICATIONS

This application claims the benefit of Chinese Patent Application No. 201910833221.X filed on Sep. 4, 2019, the disclosure of which is herein incorporated by reference in its entirety.

### TECHNICAL FIELD

The present invention relates to the field of medical imaging technologies, and in particular, to a method and system for shear wave elastography. Particularly, the present invention further relates to a computer-readable storage medium storing a computer program capable of implementing the method described above.

### BACKGROUND

Ultrasonic imaging is a medical imaging technique for forming images of internal organs and soft tissue of human bodies. In ultrasonic imaging, real-time non-invasive high-frequency sound waves are used to produce two-dimensional (2D) images and/or three-dimensional (3D) images.

Elastography is a medical imaging modality that maps elastic properties of soft tissue. Elastography can be used in medical diagnosis because it can distinguish healthy tissue from unhealthy tissue in specific organs and/or neoplasms. For example, a malignant tumor is often stiffer than the surrounding tissue, and a diseased liver is harder than a healthy liver. Elastography has been used to guide or replace biopsies by, for example, identifying potentially cancerous tissue or other diseased tissue based on tissue stiffness.

Several ultrasonic elastography techniques are known in the art. In compression-based elastography, the procedure is performed by applying external compression to tissue and comparing ultrasonic images prior to and during compression. A spectral tracking technique can be used to track tissue deformation. An image region with the least deformation has high stiffness, while a region with the greatest deformation has the lowest stiffness. Another ultrasonic elastography technique would be shear wave elastography. In shear wave elastography, thrust interference is caused in tissue through, for example, a force focusing ultrasonic beams or an external thrust. Thrust interference generates a shear wave propagating laterally from a disturbance point. An ultrasonic device acquires image data of the shear wave and determines the velocity for the shear wave to travel through different lateral positions in the tissue. An elasticity graph may be created based on the velocity of the shear wave.

Vibration frequency is critical in shear wave elastography. In conventional techniques, the vibration frequency is usually unchanging. However, at different vibration frequencies, elastic properties of the tissue may exhibit huge differences due to the factor of tissue viscosity. Thus, generating shear waves with the same vibration frequency across different clinical applications would not be appropriate. To solve this issue, clinicians would have to manually adjust the frequency by determining desired frequencies for different applications. This approach, however, is time-consuming and inefficient, and the optimal imaging frequency may not be found in the end.

### SUMMARY

The objective of the present invention is to overcome the aforementioned problems and/or other problems in the prior art. Particularly, the objective is to realize automatic determination and adjustment of an optimal vibration frequency during shear wave elastography, thereby ensuring the contrast, stability, and accuracy of elastography while reducing labor and time costs. Accordingly, an exemplary embodiment of the present invention provides a method and system for shear wave elastography and a medium storing a corresponding program.

According to an exemplary embodiment, a method for shear wave elastography is provided, the method comprising: obtaining an initial image of an object; defining a region of interest in the initial image; performing shear wave elastography on the object at a plurality of different vibration frequencies, and generating a plurality of images corresponding to the plurality of different vibration frequencies; and determining an image corresponding to a specific vibration frequency in the plurality of different vibration frequencies as an optimized image based on the region of interest.

According to another exemplary embodiment, a system for shear wave elastography is provided, the system comprising: a vibration device, configured to generate a shear wave in tissue of an object at a vibration frequency; a vibration adjusting device, configured to adjust the vibration frequency of the vibration device; an ultrasonic detection device, configured to detect the shear wave in the tissue of the object; an imaging device, configured to perform shear wave elastography according to the detected shear wave; a display, configured to display an imaged image; and a processor, configured to perform the method described above.

In the method and system according to the exemplary embodiments described above, an initial image obtained by imaging an object using any imaging means is obtained; then, a tissue region of interest is defined in the initial image; afterwards, a vibration frequency is automatically adjusted as a plurality of different frequencies to perform shear wave elastography on the object at the plurality of different vibration frequencies, and generate a plurality of images corresponding to the plurality of different vibration frequencies; and an image corresponding to a specific vibration frequency in the plurality of different frequencies is determined as an optimized image based on the region of interest. The optimized image has a significant improvement over the original image in terms of contrast and stability of imaging (especially the tissue region, relative to the peripheral region). The method and system simplify the manual adjustment operation in the imaging process as compared with the prior art, save time, and automatically determine the optimal vibration frequency so as to ensure the image quality of elastography. In addition, the method and system are easy to implement and suitable for use in small and medium-sized ultrasonic systems, and thus can be extended to a larger number of more common medical institutions. For example, the method and system are well suited to evaluating the status of a donor liver during liver transplantation, as this method and system can be applied to a compact ultrasonic device (for example, LOGIQ e of General Electric Company) and can save the space in an ICU.

Optionally, the step of determining an image corresponding to a specific vibration frequency in the plurality of different vibration frequencies as an optimized image based on the region of interest comprises: for each of the plurality of different vibration frequencies, separately calculating an average velocity of a shear wave in the region of interest in each image corresponding to each of the vibration frequencies; fitting a curve describing a frequency-velocity relationship according to each of the vibration frequencies and the corresponding average velocity; and selecting one or a plurality of vibration frequencies in the plurality of different vibration frequencies as the specific vibration frequency using the fitted curve.

Optionally, a point where the specific vibration frequency and a corresponding calculated average velocity thereof are located has a smallest distance from the fitted curve.

Optionally, the step of determining an image corresponding to a specific vibration frequency in the plurality of different vibration frequencies as an optimized image further comprises: setting a plurality of frequency windows, the plurality of frequency windows each comprising one or a plurality of vibration frequencies in the plurality of different vibration frequencies; calculating a sum of distances between points where the vibration frequencies in each frequency window and corresponding calculated average velocities thereof are located and the fitted curve; and determining a window having a smallest sum of distances in the plurality of frequency windows, wherein the specific vibration frequency is in the window. Preferably, a point where the specific vibration frequency and a corresponding calculated average velocity are located has a smallest distance from the fitted curve in the window.

Optionally, the fitting of the curve describing the frequency-velocity relationship is based on a least squares method.

Optionally, the region of interest comprises lesion tissue.

Optionally, the method or the step performed by the processor further comprises: displaying the plurality of images and marking the image corresponding to the specific vibration frequency in the plurality of displayed images.

According to yet another exemplary embodiment, a computer storage medium is further provided which stores a program executable by a computer, wherein when running, the program is capable of implementing the system and method according to the exemplary embodiments described above.

Other features and aspects will become clear through the following detailed description, accompanying drawings, and claims.

### BRIEF DESCRIPTION OF THE DRAWINGS

The present invention may be better understood by describing exemplary embodiments of the present invention with reference to accompanying drawings, in which:

FIG. 1 illustrates a basic process 100 of shear wave elastography based on a vibrator according to an exemplary embodiment of the present invention;

FIG. 2 is a flowchart of a method 200 for shear wave elastography according to an exemplary embodiment of the present invention;

FIG. 3 illustrates an image screenshot displayed on a display screen of an ultrasonic imaging device by performing shear wave elastography;

FIG. 4 illustrates a process for determining an optimized image according to an exemplary embodiment of the present invention;

FIG. 5 is a local velocity distribution graph of a shear wave in tissue generated by performing shear wave elastography using different vibration frequencies;

FIG. 6a is a graph illustrating a relationship between a shear wave velocity and a shear wave frequency that is obtained according to experimental research for different tissue;

FIG. 6b illustrates an exemplary setting manner of frequency windows according to an exemplary embodiment of the present invention;

FIG. 7 is a block diagram of a system 700 for shear wave elastography according to an exemplary embodiment of the present invention;

FIG. 8 illustrates an example of a waveform of a shear wave according to an exemplary embodiment of the present invention; and

FIG. 9 illustrates an exemplary implementation manner of a vibration adjusting device according to an exemplary embodiment of the present invention.

### DETAILED DESCRIPTION

Specific implementation manners of the present invention will be described in the following. It should be noted that during the specific description of the implementation manners, it is impossible to describe all features of the actual implementation manners in detail in this description for the sake of brief description. It should be understood that in the actual implementation of any of the implementation manners, as in the process of any engineering project or design project, a variety of specific decisions are often made in order to achieve the developer's specific objectives and meet system-related or business-related restrictions, which will vary from one implementation manner to another. Moreover, it can also be understood that although the efforts made in such development process may be complex and lengthy, for those of ordinary skill in the art related to content disclosed in the present invention, some changes in design, manufacturing, production or the like based on the technical content disclosed in the present disclosure are only conventional technical means, and should not be construed as that the content of the present disclosure is insufficient.

Unless otherwise defined, the technical or scientific terms used in the claims and the description are as they are usually understood by those of ordinary skill in the art to which the present invention pertains. The words “first,” “second” and similar words used in the description and claims of the patent application of the present invention do not denote any order, quantity or importance, but are merely intended to distinguish between different constituents. The terms “one,” “a/an,” and the like do not denote a limitation of quantity, but rather denote the presence of at least one of the referenced items. The terms “include,” “comprise,” and the like are intended to mean that an element or article that appears before “include” or “comprise” encompasses elements or articles and equivalent elements that are listed after “include” or “comprise,” and do not exclude other elements or articles. The word “connect,” “connected” or a similar word is not limited to a physical or mechanical connection, and is not limited to a direct or indirect connection.

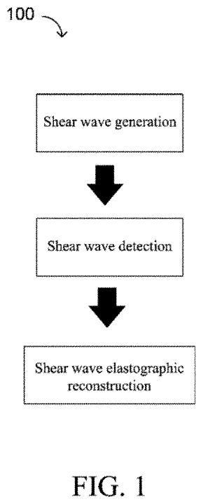

FIG. 1 illustrates a basic process 100 of shear wave elastography based on a vibrator according to an exemplary embodiment of the present invention.

As shown in FIG. 1, first, a vibrator (for example, a linear motor) is used to generate a shear wave at a vibration frequency and direct the shear wave into tissue to be imaged (namely, “shear wave generation”). The shear wave is a transverse wave. The propagation velocity of shear waves in human tissue is approximately 1 to 10 meters/second in medical applications. The vibrator may be disposed outside an ultrasonic detection device to serve as an external vibrator, or may be disposed inside the ultrasonic detection device to serve as an internal vibration source. The shear wave may be generated by mechanical vibration or may be excited at a preset position by an acoustic radiation force. Then, the shear wave is detected using an acoustic beam sequence (namely, “shear wave detection”). For example, an ultrasonic system may be used to acquire shear wave ultrasonic data from the tissue to be imaged at a high pulse repetition frequency. Finally, an elasticity or viscosity graph of the tissue is reconstructed from the detected shear wave data using an algorithm (namely, “shear wave elastographic reconstruction”). For example, a processor may be used to process the shear wave (ultrasonic) data to determine the local velocity distribution of the shear wave passing through the tissue to be imaged. Specifically, the shear wave velocity of the shear wave (ultrasonic) data at each position may be calculated through direct inversion of a Helmholtz equation, time-of-flight measurement, or any suitable calculation method. Afterwards, the determined local velocity distribution of the shear wave may be converted into a graph. In various embodiments, the graph may be a velocity distribution graph, an elasticity graph, a viscosity graph, a spatial gradient graph, or any suitable graph representing a contrast between different tissue. For example, the local distribution may be mapped based on the shear wave velocity to generate a velocity distribution graph. As another example, the local distribution may be converted into an elasticity graph by calculating stiffness based on Young's modulus, a similar shear modulus, or any suitable conversion calculation. In addition, a spatial gradient filter may be applied to the velocity distribution graph and/or elasticity graph to generate a spatial gradient graph. The graph may be a color-coded graph or gray-scale graph having different colors or gray-scales corresponding to different velocities and/or elasticities. For example, the color-coded graph or gray-scale elasticity graph may display soft tissue in a dark color, while tissue having greater stiffness than the soft tissue may be displayed in a light color, and so on.

Vibration frequency is critical in shear wave elastography. In conventional techniques, the vibration frequency is usually unchanging, and shear wave detection is performed using a relatively large packet size. However, at different vibration frequencies, elastic properties of the tissue may exhibit huge differences due to the factor of tissue viscosity. Thus, generating shear waves with the same vibration frequency across different clinical applications would not be appropriate. To solve this issue, clinicians would have to manually adjust the frequency by determining desired frequencies for different applications. This approach, however, is time-consuming and inefficient, and the optimal imaging frequency may not be found in the end.

A method for shear wave elastography provided according to an embodiment of the present invention is described in detail below with reference to the accompanying drawings.

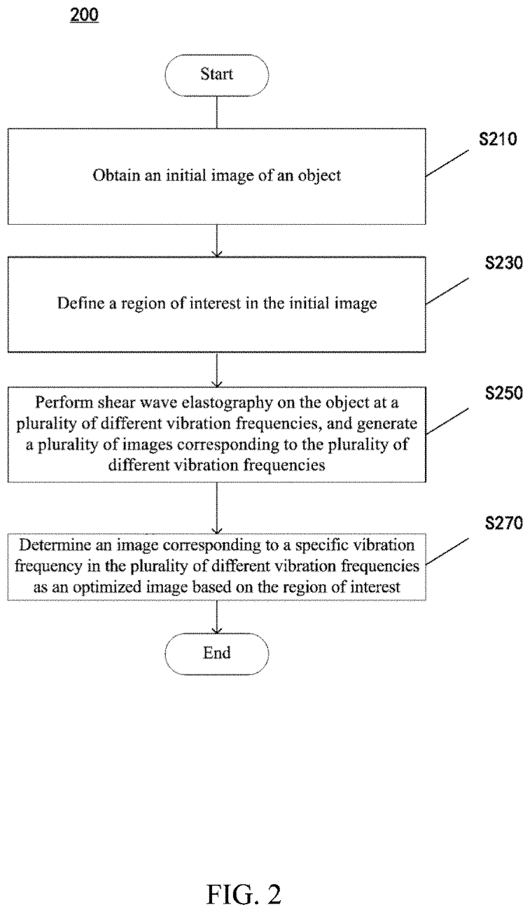

Referring to FIG. 2, FIG. 2 is a flowchart of a method 200 for shear wave elastography according to an exemplary embodiment of the present invention. As shown in FIG. 2, the method 200 for shear wave elastography according to an exemplary embodiment of the present invention may include the following steps S210 to S270.

Step S210: obtain an initial image of an object.

The initial image of the object may come from any imaging system, and may be generated by any imaging means. The initial image of the object may be generated in real time by any imaging system, or may be stored in a memory, and the imaging system for generating the initial image or the memory storing the initial image can be accessed to obtain the initial image of the object. For example, in some embodiments of the present invention, an ultrasonic imaging device may be used to perform ordinary 2D or 3D ultrasonic imaging on the object to generate the initial image, or the ultrasonic imaging device may be used to perform the shear wave elastography process described with reference to FIG. 1 on the object to generate the initial image at an initial vibration frequency. The initial vibration frequency may be from clinical test feedback, for example, set to 100 Hz. Note that the initial vibration frequency may also be selected in other manners, or the initial vibration frequency may be arbitrarily set.

Step S230: define a region of interest in the initial image.

The region of interest of the image may be defined by a user or automatically set by a system. In some embodiments of the present invention, after the initial image of the object is obtained, the initial image may be displayed on a display screen for viewing by the user. If the image has a region that the user expects to focus on, the user may set the region as the region of interest. For example, the region of interest of the image may include tissue suspected to be a lesion. The region of interest may be of any shape, for example, a circle.

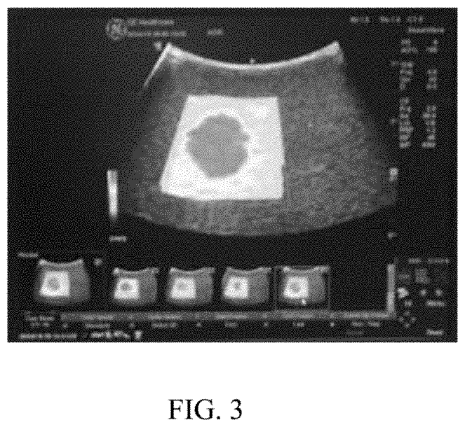

As an example, referring to FIG. 3, FIG. 3 illustrates a reconstructed image imaged using the ultrasonic imaging device and displayed on the display screen of the ultrasonic imaging device. After viewing the reconstructed image, the user may set the region of interest, for example, a circular region in the image displayed in FIG. 3, through an input apparatus of the ultrasonic imaging device.

Referring back to FIG. 2, step S250: perform shear wave elastography on the object at a plurality of different vibration frequencies, and generate a plurality of images corresponding to the plurality of different vibration frequencies. In some embodiments of the present invention, real-time shear wave elastography performed using a plurality of different vibration frequencies may be implemented using the process described with reference to FIG. 1, and a plurality of images corresponding to the plurality of different vibration frequencies are generated during the real-time shear wave elastography. For example, after the region of interest is defined, shear wave elastography may be performed by adjusting the vibration frequency as one or a plurality of different values, so as to obtain images corresponding to the different vibration frequencies.

In some embodiments of the present invention, shear wave elastography is performed using different vibration frequencies, so that the vibration frequency can be gradually adjusted from a minimum to a maximum (or the vibration frequency can be gradually adjusted from the maximum to the minimum) within a frequency range, and shear wave ultrasonic data can be acquired in real time to obtain the shear wave velocity distribution. The frequency range may be any frequency range between the minimum vibration frequency and the maximum vibration frequency that the vibrator can achieve.

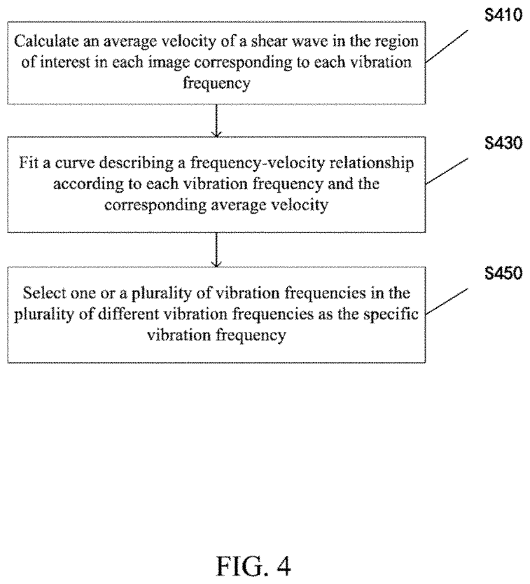

Step S270: determine an image corresponding to a specific vibration frequency in the plurality of different vibration frequencies as an optimized image. The optimized image has a significant improvement over the original image in terms of contrast and stability of imaging. In addition, as described in detail below, the accuracy of elastography is also improved as non-zero viscous interference is eliminated. In some embodiments of the present invention, step S270 may include steps S410 to S450, as shown in FIG. 4.

Step S410: for each of the plurality of different vibration frequencies, separately calculate an average velocity of a shear wave in the region of interest in each image corresponding to each of the vibration frequencies.

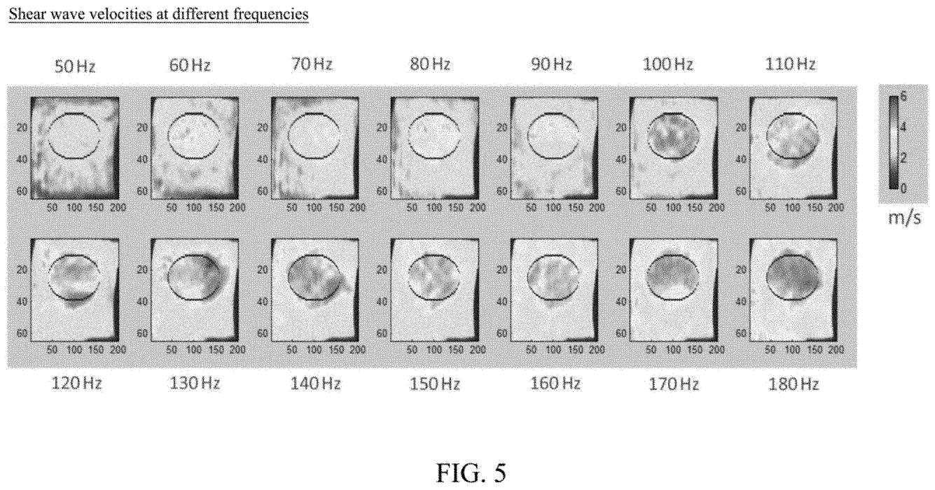

Referring to FIG. 5, FIG. 5 is a local velocity distribution graph of a shear wave in tissue generated by performing shear wave elastography using different vibration frequencies. As shown in FIG. 5, the region of interest of the image is set after the initial image is obtained, and then an average velocity of a shear wave in the region of interest of a corresponding image may be calculated for each frequency.

Referring back to FIG. 4, step S430: fit a curve describing a frequency-velocity relationship according to each vibration frequency and the corresponding average velocity.

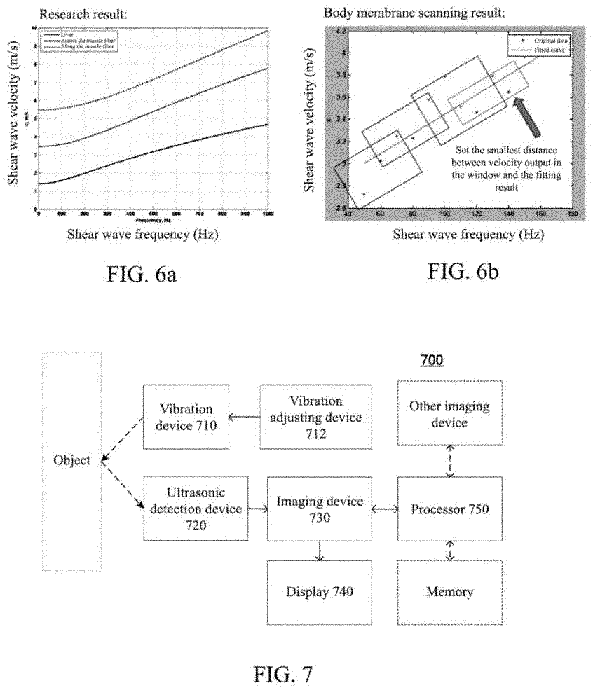

The velocity of the shear wave is related to the vibration frequency, and they are usually non-linearly related. Referring to FIG. 6a, FIG. 6a is a graph illustrating a relationship between a shear wave velocity and a shear wave frequency that is obtained according to experimental research for different tissue. FIG. 6a illustrates curves of relationships between shear wave velocities and shear wave frequencies in the liver, across the muscle fiber, and along the muscle fiber respectively. In some embodiments of the present invention, the curve describing the frequency-velocity relationship may be fitted according to each vibration frequency and the corresponding average velocity using any fitting algorithm (such as a least squares method).

Step S450: select one or a plurality of vibration frequencies in the plurality of different vibration frequencies as the specific vibration frequency using the fitted curve. The curve describing the frequency-velocity relationship is generally affected by both a viscosity parameter and an elasticity parameter of the tissue. In order to minimize the influence of the viscosity parameter on the shear wave velocity and obtain the most accurate tissue elasticity graph, it is necessary to identify an optimal or preferred frequency. The optimal or preferred vibration frequency may be determined through an algorithm. The optimal vibration frequency may be defined to have the smallest distance between the fitted curve and a point where original data is located, and the preferred vibration frequencies may be defined to have relatively small distances between the fitted curve and a plurality of points where original data is located (namely, have smaller distances from the fitted curve as compared with other points than the plurality of points). In this way, the influence of the viscosity parameter on the shear wave velocity can be estimated by performing fitting in the most precise manner, thereby selecting a specific vibration frequency to minimize the influence of the viscosity parameter on the shear wave velocity. In other words, the accuracy of elastography can be improved as non-zero viscous interference is basically eliminated.

During the aforementioned shear wave elastography, some random noise may exist, which may influence the calculation result of the shear wave velocity at the specific frequency. Thus, in the aforementioned step of determining the optimal or preferred vibration frequency, if the distance between the fitted curve and a point where original data (namely, a frequency and a corresponding calculated average velocity) is located is checked only for a single frequency at a time, an improper vibration frequency may be selected as the specific vibration frequency for imaging. For example, an improper vibration frequency may be determined as the optimal or preferred vibration frequency under the interference of random noise. In view of this, optionally, the aforementioned step S270 of determining an image corresponding to a specific vibration frequency in the plurality of different vibration frequencies as an optimized image may further include the following steps: setting a plurality of frequency windows, the plurality of frequency windows each comprising one or a plurality of vibration frequencies in the plurality of different vibration frequencies; calculating a sum of distances between points where the vibration frequencies in each frequency window and corresponding calculated average velocities thereof are located and the fitted curve; and determining a window having a smallest sum of distances in the plurality of frequency windows, wherein the specific vibration frequency is in the window.

In some embodiments of the present invention, a series of frequency windows may be set, so as to separately determine, for these frequency windows, a sum of distances between a fitted curve of a plurality of vibration frequencies therein and points where corresponding original data is located, and identify an optimal frequency window by judging which frequency window has a smallest sum of distances. In this way, any one (or a plurality of) vibration frequencies may be selected in the optimal frequency window to serve as the optimal (or preferred) vibration frequency. For example, in the optimal frequency window, a point where the optimal vibration frequency and a corresponding calculated average velocity thereof are located may have the smallest distance from the fitted curve, while a plurality of points where a plurality of preferred vibration frequencies and corresponding calculated average velocities thereof are located may have relatively small distances from the fitted curve (namely, have smaller distances from the fitted curve as compared with other points than the plurality of points in the window).

The frequency windows may be set in a variety of manners so that a series of frequency windows each include a plurality of consecutive frequencies in a plurality of different vibration frequencies, These frequency windows are different from each other, but can share some identical vibration frequencies. Referring to FIG. 6b, FIG. 6b illustrates an exemplary setting manner of frequency windows. FIG. 6b depicts four frequency windows and ten pieces of original data (imaging frequencies and corresponding velocities). The four frequency windows each include three or four pieces of original data. For each frequency window, distances between a fitting result of vibration frequencies therein and original velocities are separately calculated and summed, a frequency window having the smallest sum of distances (the window pointed to by the arrow in FIG. 6) is determined as the optimal, and then an optimized vibration frequency is selected therefrom. Note that the present invention is not intended to limit the setting manner (for example, the size of the frequency window and the number of frequencies contained). The frequency window may also be set through a specific frequency interval, a specific velocity interval, a specific frequency quantity, and so on.

The method for shear wave elastography according to the exemplary embodiment of the present invention is described above. By means of the method, an initial image obtained by imaging an object using any imaging means is obtained; then, a tissue region of interest is defined in the initial image; afterwards, a vibration frequency is automatically adjusted as a plurality of different frequencies to perform shear wave elastography on the object at the plurality of different vibration frequencies, and generate a plurality of images corresponding to the plurality of different vibration frequencies; and an image corresponding to a specific vibration frequency in the plurality of different frequencies is determined as an optimized image based on the region of interest. The optimized image has a significant improvement over the original image in terms of contrast and stability of imaging (especially the tissue region, relative to the peripheral region). The method simplifies the manual adjustment operation in the imaging process as compared with the prior art, saves time, and automatically determines the optimal vibration frequency so as to ensure the image quality of elastography. In addition, the method is easy to implement and suitable for use in small and medium-sized ultrasonic systems, and thus can be extended to a larger number of more common medical institutions. For example, the method is well suited to evaluating the status of a donor liver during liver transplantation, as this method can be applied to a compact ultrasonic device (for example, LOGIQ e of General Electric Company) and can save the space in an ICU.

Optionally, the plurality of previously generated images corresponding to a plurality of different frequencies may be displayed to a user, and the image corresponding to the specific frequency may be marked. For example, the plurality of images corresponding to a plurality of different frequencies shown in FIG. 5 may be displayed on a display, and then an image corresponding to an optimal or preferred frequency automatically selected according to the method of the present invention may be marked for reference by the user. In this way, a physician can compare a plurality of images corresponding to different frequencies and judge whether the imaging quality of an automatically selected image meets his expectation. If yes, the physician may use the automatically selected image for subsequent diagnosis. If not, the physician may select other images for subsequent diagnosis.

Like the aforementioned method, the present invention further provides a corresponding system.

FIG. 7 is a block diagram of a system 700 for shear wave elastography according to an exemplary embodiment of the present invention. The system 700 includes: a vibration device 710, configured to generate a shear wave in tissue of an object at a vibration frequency; a vibration adjusting device 712, configured to adjust the vibration frequency of the vibration device; an ultrasonic detection device 720, configured to detect the shear wave in the tissue of the object; an imaging device 730, configured to perform shear wave elastography according to the detected shear wave; a display 740, configured to display an imaged image; and a processor 750, configured to perform the method (namely, each step) described above. For example, the processor 750 may obtain an initial image of the object from the imaging device 730 or may communicate with any other imaging device or a memory (indicated by dashed boxes) to obtain the initial image of the object, and then perform subsequent steps of the method of the present invention.

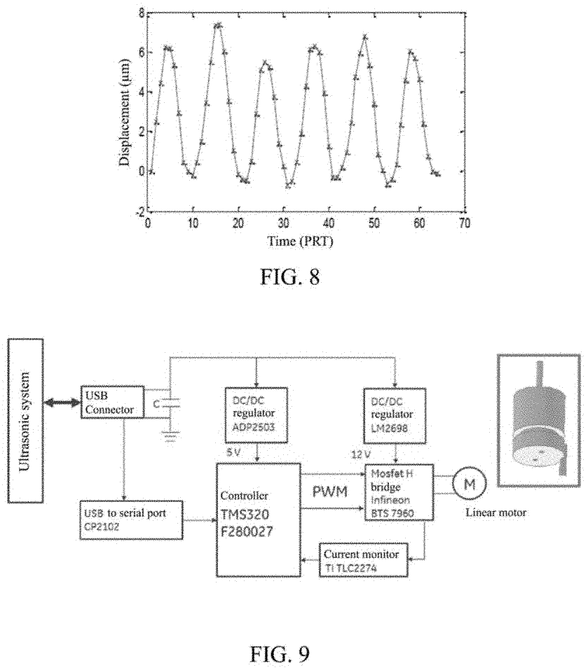

FIG. 8 illustrates an example of a waveform of a shear wave according to an exemplary embodiment of the present invention. FIG. 9 illustrates an exemplary implementation manner of the vibration adjusting device. A DSP control signal chain of the vibration adjusting device shown in FIG. 9 may be used to adjust in real time the waveform of the shear wave generated by the vibration device, for example, change the output frequency and amplitude of the vibration device in real time.

The system for shear wave elastography according to the exemplary embodiment of the present invention is described above. By means of the system, an initial image obtained by imaging an object using any imaging means is obtained; then, a tissue region of interest is defined in the initial image; afterwards, a vibration frequency is automatically adjusted as a plurality of different frequencies to perform shear wave elastography on the object at the plurality of different vibration frequencies, and generate a plurality of images corresponding to the plurality of different vibration frequencies; and an image corresponding to a specific vibration frequency in the plurality of different frequencies is determined as an optimized image based on the region of interest. The optimized image has a significant improvement over the original image in terms of contrast and stability of imaging (especially the tissue region, relative to the peripheral region). The system simplifies the manual adjustment operation in the imaging process as compared with an existing system, saves time, and automatically determines the optimal vibration frequency so as to ensure the image quality of elastography. In addition, the system is easy to implement and suitable for implementation as small and medium-sized ultrasonic systems, and thus can be extended to a larger number of more common medical institutions. For example, the system is well suited to evaluating the status of a donor liver during liver transplantation, as this system can be implemented as a compact ultrasonic device (for example, LOGIQ e of General Electric Company) and can save the space in an ICU.

An exemplary model describing a frequency-velocity relationship of a shear wave is introduced below.

A relationship between a velocity of a shear wave and a vibration frequency thereof may be described using a viscoelasticity model (namely, through an elasticity parameter and a viscosity parameter). An example of the viscoelasticity model is a Voigt model whose expression is as follows:

$${c}_{s} ( ω ) = \sqrt{\frac{2 ( {μ}_{1}^{2} + {ω}^{2} {μ}_{2}^{2} )}{ρ ( {μ}_{1} + \sqrt{{μ}_{1}^{2} + {ω}^{2} {μ}_{2}^{2}} )}} ,$$

where co is the shear wave frequency, cₛ is the shear wave velocity, μ₁ is the elasticity parameter, μ₂ is the viscosity parameter, and p is a constant greater than zero. In some embodiments of the present invention, p may be set to 1. Note that the Voigt model simply describes an exemplary model of the relationship between the velocity of the shear wave and the vibration frequency thereof, and the present invention is not intended to limit the form of the viscoelasticity model. In general practice, for example, in the actual operation previously issued, the viscosity parameter μ₂ is assumed to be zero, and thus the shear wave velocity is only related to the elasticity parameter μ₁. However, such assumption is inappropriate for the actual situation in many cases, as the viscosity of the tissue does exist and cannot be ignored. A quick and effective way to verify the existence of the tissue viscosity and affect the shear wave velocity is to adjust the vibration frequency and then check whether a velocity distribution graph (from which an elasticity graph is created) changes accordingly. If a significant change is observed, it can be concluded that the viscosity cannot be ignored and does affect the shear wave velocity.

In some embodiments of the present invention, after shear wave elastography is performed using different vibration frequencies and average velocities of a shear wave in a region of interest corresponding to the different frequencies is obtained, these frequencies and corresponding average velocities of the shear wave are fitted into a viscoelasticity model (for example, the aforementioned Voigt model), so as to obtain an elasticity parameter and a viscosity parameter in the viscoelasticity model (for example, μ₁ and μ₂ in the Voigt model). The fitting algorithm may be a least squares method or any other fitting calculation method.

As described above, in order to minimize the influence of the viscosity parameter (μ₂) on the shear wave velocity and obtain the most accurate tissue elasticity graph, it is necessary to identify an optimal or preferred frequency. The optimal vibration frequency may be defined to have the smallest distance between the curve fitted based on the viscoelasticity model (for example, the Voigt model) and a point where original data is located, and the preferred vibration frequencies may be defined to have relatively small distances between the curve fitted based on the viscoelasticity model (for example, the Voigt model) and a plurality of points where original data is located (namely, have smaller distances from the fitted curve as compared with other points than the plurality of points). In this way, the influence of the viscosity parameter (μ₂) on the shear wave velocity can be estimated by performing fitting in the most precise manner, thereby selecting a specific vibration frequency to minimize the influence of the viscosity parameter (μ₂) on the shear wave velocity.

The technique described herein may be implemented with hardware, software, firmware, or any combination thereof, unless specifically described as being implemented in a specific manner. Any features described as modules or components may also be implemented together in an integrated logical apparatus, or separately implemented as discrete but interoperable logical apparatuses. If implemented with software, the technique may be implemented at least in part by a non-transitory processor-readable storage medium that includes instructions, where when executed, the instructions perform one or more of the aforementioned methods. The non-transitory processor-readable data storage medium may form part of a computer program product that may include an encapsulation material. Program code may be implemented in a high-level procedural programming language or an object-oriented programming language so as to communicate with a processing system. If desired, the program code may also be implemented in an assembly language or a machine language. In fact, the mechanisms described herein are not limited to the scope of any particular programming language. In any case, the language may be a compiled language or an interpreted language.

One or a plurality of aspects of at least some embodiments may be implemented by representative instructions that are stored in a machine-readable medium and represent various logic in a processor, where when read by a machine, the representative instructions cause the machine to manufacture the logic for executing the technique described herein.

Such computer-readable storage medium may include, but is not limited to, a non-transitory, tangible arrangement of an article manufactured or formed by a machine or apparatus, including a storage medium such as a hard disk; any other type of disk including a floppy disk, an optical disk, a compact disk read-only memory (CD-ROM), a compact disk rewritable (CD-RW), and a magneto-optical disk; a semiconductor device such as a read-only memory (ROM), a random access memory (RAM) such as a dynamic random access memory (DRAM) and a static random access memory (SRAM), an erasable programmable read-only memory (EPROM), a flash memory, and an electrically erasable programmable read-only memory (EEPROM); a phase change memory (PCM); a magnetic or optical card; or any other type of medium suitable for storing electronic instructions.

Instructions may further be sent or received via a network interface apparatus that uses any of a number of transport protocols (for example, Frame Relay, Internet Protocol (IP), Transfer Control Protocol (TCP), User Datagram Protocol (UDP), and Hypertext Transfer Protocol (HTTP)) and through a communication network using a transmission medium.

An exemplary communication network may include a local area network (LAN), a wide area network (WAN), a packet data network (for example, the Internet), a mobile phone network (for example, a cellular network), a plain old telephone service (POTS) network, and a wireless data network (for example, Institute of Electrical and Electronics Engineers (IEEE) 802.11 standards referred to as Wi-Fi®, and IEEE 802.16 standards referred to as WiMax®), IEEE 802.15.4 standards, a peer-to-peer (P2P) network, and the like. In an example, the network interface apparatus may include one or a plurality of physical jacks (for example, Ethernet, coaxial, or phone jacks) or one or a plurality of antennas for connection to the communication network. In an example, the network interface apparatus may include a plurality of antennas that wirelessly communicate using at least one technique of single-input multiple-output (SIMO), multiple-input multiple-output (MIMO), or multiple-input single-output (MISO) techniques.

The term “transmission medium” should be considered to include any intangible medium capable of storing, encoding, or carrying instructions for execution by a machine, and the “transmission medium” includes digital or analog communication signals or any other intangible medium for facilitating communication of such software.

By now, a method and system for shear wave elastography according to the present invention have been described, and a computer-readable storage medium capable of implementing the method has also been introduced.

Some exemplary embodiments have been described above. However, it should be understood that various modifications can be made to the exemplary embodiments described above without departing from the spirit and scope of the present invention. For example, an appropriate result can be achieved if the described techniques are performed in a different order and/or if the components of the described system, architecture, apparatus, or circuit are combined in a different manner and/or replaced or supplemented with additional components or equivalents thereof; accordingly, the modified other embodiments also fall within the protection scope of the claims.

1. A method for shear wave elastography, the method comprising:
obtaining an initial image of an object;
defining a region of interest in the initial image;
performing shear wave elastography on the object at a plurality of different vibration frequencies, and generating a plurality of images corresponding to the plurality of different vibration frequencies; and
determining an image corresponding to a specific vibration frequency in the plurality of different vibration frequencies as an optimized image based on the region of interest.

2. The method according to claim 1, wherein the step of determining an image corresponding to a specific vibration frequency in the plurality of different vibration frequencies as an optimized image based on the region of interest comprises:
for each of the plurality of different vibration frequencies, separately calculating an average velocity of a shear wave in the region of interest in each image corresponding to each of the vibration frequencies;
fitting a curve describing a frequency-velocity relationship according to each of the vibration frequencies and the corresponding average velocity; and
selecting one or a plurality of vibration frequencies in the plurality of different vibration frequencies as the specific vibration frequency using the fitted curve.

3. The method according to claim 2, wherein a point where the specific vibration frequency and a corresponding calculated average velocity thereof are located has a smallest distance from the fitted curve.

4. The method according to claim 2, wherein the step of determining an image corresponding to a specific vibration frequency in the plurality of different vibration frequencies as an optimized image further comprises:
setting a plurality of frequency windows, the plurality of frequency windows each comprising one or a plurality of vibration frequencies in the plurality of different vibration frequencies;
calculating a sum of distances between points where the vibration frequencies in each frequency window and corresponding calculated average velocities thereof are located and the fitted curve; and
determining a window having a smallest sum of distances in the plurality of frequency windows, wherein
the specific vibration frequency is in the window.

5. The method according to claim 4, wherein a point where the specific vibration frequency and a corresponding calculated average velocity are located has a smallest distance from the fitted curve in the window.

6. The method according to claim 2, wherein the fitting of the curve describing the frequency-velocity relationship is based on a least squares method.

7. The method according to claim 1, wherein the region of interest comprises lesion tissue.

8. The method according to claim 1, wherein the method further comprises: displaying the plurality of images and marking the image corresponding to the specific vibration frequency in the plurality of displayed images.

9. A computer-readable storage medium storing a computer program, wherein when executed by a processor, the program implements the steps of the method according to claim 1.

10. A system for shear wave elastography, the system comprising:
a vibration device, configured to generate a shear wave in tissue of an object at a vibration frequency;
a vibration adjusting device, configured to adjust the vibration frequency of the vibration device;
an ultrasonic detection device, configured to detect the shear wave in the tissue of the object;
an imaging device, configured to perform shear wave elastography according to the detected shear wave;
a display, configured to display an imaged image; and
a processor, configured to perform the method according to claim 1.
